(19) United States
(12) Patent Application Publication
(10) Pub. No.: US 2009/0076413 A1
(43) Pub. Date: Mar. 19, 2009

(54) DEVICE AND SYSTEM FOR SPECIMEN COLLECTION

(76) Inventor: Angela L. Robles, Winnetka, CA (US)

Correspondence Address: SEYFARTH SHAW LLP, 131 S. DEARBORN ST., SUITE 2400, CHICAGO, IL, 60603-5803, US

(21) Appl. No.: 12/194,281

(22) Filed: Aug. 19, 2008

Related U.S. Application Data
(60) Provisional application No. 60/965,419, filed on Aug. 20, 2007.

(51) Int. Cl. A61B 5/20

(52) U.S. Cl. 600/573; 604/317

(57) ABSTRACT

Holders for specimen collection such as urine collection and systems including holders and specimen collection cups are disclosed. The holders include resiliently deformable features for retaining cups of varying sizes therein. In some forms, the holders are alignable to minimize spillage when advancing the specimen cup into a urine stream.

### CROSS-REFERENCE TO RELATED APPLICATIONS(S)

This application claims the benefit of U.S. Provisional Patent Application No. 60/965,419, filed Aug. 20, 2007, titled “Device for Assisting in Urine Collection,” the entirety of which is incorporated herein by reference.

### FIELD OF THE INVENTION

The invention relates to the collection of urinary specimens and, in particular, to systems and devices providing handles for manipulating a specimen collection cup during collection of a urinary specimen.

### BACKGROUND

There are many medical purposes for collecting specimens and, in particular, collecting urine or urinary specimens from patients or persons. Generally speaking, the urine may easily be analyzed to determine the composition thereof, which itself provides an indication of many health and physiological characteristics of the patient, such a process referred to as urinalysis. For example, many athletes provide specimens or samples for urinalysis that demonstrate or confirm the athlete's suitability for participation in sports, that is, that the athlete is healthy and free of drugs such as performance enhancing or illegal drugs. Pregnant women will provide specimens or samples so that their body's chemistry can be monitored to protect the health of the woman and the unborn child, such as identify gestational diabetes.

Generally speaking, the typical manner in which a urine specimen is collected is simply providing the patient with a specimen cup into which the patient is to deposit their urine. While usually the patient is allowed to visit a restroom for privacy in collecting the specimen, some patients do require assistance in collection. Again, in simplest terms, the patient holds or positions the specimen cup proximate but a short distance away from their urethral opening to collect urine being released therefrom.

For a variety of reasons, it is common for the patient to be instructed to collect a mid-stream portion. That is, the patient is to urinate for a short period of time into a toilet, then, for a period of time, position the specimen cup in a position to collect a sufficient amount of the urine stream. Once the desired amount of urine is collected in the specimen cup, the patient is to remove the specimen cup from the urine stream so that the voiding or remainder of the patient's urine is again directed into the toilet.

For all people there are issues attendant to this process and, for a variety of people having certain physical characteristics, there are additional issues presented. In general, it is difficult (if not impossible) to collect a midstream portion of the urine without having some portion thereof splash the edge and side of the specimen cup. Some patients may be able to contract their muscles to stop the flow of urination, but some of these patients may have trouble restarting due to the relief on the bladder from the initial urine that has already been voided. Beyond the medical reasons for midstream collection, such as allows the patient to clearly identify where the specimen cup should be positioned, as opposed to aiming one's urine stream prior to beginning urination. Generally speaking, the specimen cup is positioned a distance away from the urethral opening. This reduces contamination of the urine specimen by contact with the patient's body, and this reduces the likelihood of splashing.

In any event, it is most common for some portion of the urine to be on the outside of the specimen cup. Both patients and medical personnel prefer to avoid having to come in contact with the patients' urine. In any event, the filled specimen cup is typically closed with a snap-on or screw-on lid, and the specimen cup is labeled for the patient's identifying information. In order to secure the lid thereon, the specimen cup and lid must be manually manipulated. The label may be affixed to the specimen cup prior to filling, in which case the label information may have become wet and run or the label itself may have absorbed urine, or may be affixed after filling which again requires manual manipulation.

Adding to the general difficulty of maintaining a clean exterior for the specimen cup are particular physical issues for a variety of people. That is, many people present additional difficulties due to their own physicality. For instance, many people such as pregnant women and obese people find it difficult to properly locate the specimen cup proximate the urethral opening. Many people find it difficult to properly position the cup during midstream collection, such as some elderly men who tend to urinate in short bursts as opposed to a continuous stream.

A number of attempts have been made to provide devices that assist in collection of urine. A basic specimen cup holder is shown in U.S. Design Pat. No. D399,007 (“the '007 patent”). As shown, the depicted device is an elongated handle with a support ring for receiving a specimen cup. The size of the cup is accommodating by the device only by the tapered cup being received to the degree that such can be advanced into the support ring.

Somewhat of an improvement upon the '007 patent is U.S. Pat. No. 5,492,220 (“the '220 patent”) which also describes an elongated handle having a distally formed ring thereon for supporting a tapered specimen cup. The ring includes an opening or break positioned to a side, i.e., not along the longitudinal axis of the device, the opening allowing some flexure of the ring to accommodate different sized specimen cups. However, it should be obvious that a larger cup would either sit high in the ring, akin to what is shown in FIG. 1 of the '220 patent, or that the cup would be pressed into the ring. In the latter case, the only manner in which the ring could avoid exerting a substantial spring force against cup is for the ring to be of a sufficiently soft material to allow for significant expansion of the ring; in this case, the ring may then be inadequately stiff for holding the specimen cups once they become weighed with collected urine.

U.S. Design Pat. No. D379,655 (“the '655 patent”) shows an elongated handle and distally formed ring supporting a cup, each provided with a complementary sliding component allowing manipulation of a cup cover via a handle portion. As stated above, it is most typical for a filled specimen cup to be closed for processing. For the '655 patent, it is clear that the cup cannot be removed from the handle without also removing the lid, if at all. Therefore, it is clear that the assembly is meant to be transported to a laboratory for analysis in its entirety. Doing so is obviously awkward and inconvenient, and the protruding handles make the assembly prone to being accidentally bumped.

U.S. Pat. No. 6,602,231 (“the '231 patent”) shows a collection cup having a lid thereon and having a telescoping handle. Again, like the '655 patent, the '231 patent does not allow the handle to be removed from the cup. Because the handle is telescoping, it can at least be partially collapsed to ease transport to and use in the laboratory.

U.S. Pat. No. 7,128,352 (“the '352 patent”) shows devices having an elongated handle, a ring integral with the handle, and a cup supportable by ring. Such devices are similar to and suffer from the same deficiencies in accommodating cups of different sizes as the devices of the '220 and '007 patents. Additionally, the '352 patent shows an embodiment in FIG. 16 generally formed of wire to have an open end that can be shaped to receive a desired specimen cup and to provide a desired spring compression thereto.

Accordingly, there has been a need for improved methods and devices for collecting urine.

### SUMMARY

In accordance with an aspect a device for utilizing a specimen collection cup is disclosed, the device comprising a handle portion, a support portion connected with the handle portion and defining a closed boundary, and retention features, wherein the support portion is configured to receive cups of varying sizes within the closed boundary, and wherein the retention features provide a resilient retention force to the cup received therein.

In some forms, the support portion defines a circular closed boundary and is generally ring-shaped.

In some forms, the retention features include a resiliently deformable portion extending inwardly into the closed boundary. The resiliently deformable portion may include a plurality of fingers. The fingers may be formed integral with the support portion. The fingers may taper from a base portion at the support portion to a tip portion to provide variable retention force to the varying sized cups. The support portion may be in the form of a ring, the fingers may have an arcuate base connected with the support portion, and the fingers may have an arcuate tip portion for contacting and providing retention force against a cup received within the support portion.

In some forms, the device is formed integral.

In another aspect, a holder for supporting and manipulating a specimen cup for collecting urine is disclosed, the holder including a support portion for supporting a specimen cup, an elongated handle extending from the support portion, and a frangible breakline for removing at least a portion of the handle from the support portion.

In some forms, the breakline is frangible by applying a lateral force.

In some forms, the breakline is frangible by applying a vertical force.

In some forms, the holder further includes a specimen cup. The specimen cup may be integral with the support portion.

In another aspect, a holder for supporting and manipulating a specimen cup for collecting urine is disclosed, the holder comprising an elongated handle for directing the holder, and a rigid support portion connected with the handle, wherein the support portion includes first and second arms defining an open boundary including an opening, the open boundary for receiving cups therein, and the arms including terminal portions for retaining the cups therein.

In some forms, the opening is opposite the handle. The opening may be at least a quarter of the boundary.

In some forms, the arms are generally planar and the terminal portions thereof are angled to prevent cups from passing through the opening.

In some forms, the terminal portions extend upwardly from a general plane of the arms, the terminal portions preventing cups from passing through the opening.

In another aspect, a holder for supporting and manipulating a specimen cup for collecting urine is disclosed, the holder including an elongated handle for directing the holder, and a connector formed on a distal end of the holder, the connector releasably securable with a portion of a specimen cup.

In some forms, the connector includes a pair of walls defining a channel, and a top portion of the cup is receivable between the walls. The walls may exert a friction force against a cup received therebetween, the friction force retaining the cup with the holder. The walls may include securing structure for retaining the connector with the cup. The securing structure may be at least a riblike structure for pressing into the cup. The securing structure may be at least a riblike structure for providing an interference fit with a portion of the cup.

In some forms, the connector and cup have corresponding mating structure forming a post-and-socket joint. The joint may include a horizontal socket and a post received horizontally therein. The joint may include a vertical socket and a post received vertically therein.

In another aspect, a holder for supporting and manipulating a specimen cup for collecting urine is disclosed, the holder including an elongated handle for directing the holder, a support portion for receiving a specimen cup, and a funnel positionable over and into a specimen cup received by the support portion for directing urine into the cup.

In another aspect, a holder for supporting and manipulating a specimen cup for collecting urine is disclosed, the holder including an elongated handle for directing the holder, a support portion for receiving a specimen cup, and a threaded engagement between the support portion and the specimen cup. In some forms, the holder further includes an absorbent material disposed proximate the threaded engagement.

In another aspect, a holder for supporting and manipulating a specimen cup for collecting urine is disclosed, the holder including an elongated handle for directing the holder, a support portion for receiving a specimen cup, wherein the support portion includes a generally funnel-shape for directing urine into a cup supported thereby. In some forms, the support portion may include a plurality of inwardly extending retention fingers for supporting a cup therein, the fingers separated by gaps permitting urine to pass therethrough.

In another aspect, a holder for supporting and manipulating a specimen cup for collecting urine is disclosed, the holder including a support portion for supporting a specimen cup, and first and second arms extending from the support portion and supportable by a toilet.

In some forms, the first and second arms may include widened end portions for resisting movement of the holder. The widened end portions may include fork-like portions.

In some forms, the support portion includes the specimen cup.

### BRIEF DESCRIPTION OF THE DRAWINGS

In the Figures, FIG. 1 is a perspective view of first form of a holder for supporting a specimen cup during specimen collection showing retention features formed on the holder in a first position;

FIG. 2 is a top plan view of the holder of FIG. 1 showing the retention features extending radially inward from an inner perimeter of a supporting ring of the holder;

FIG. 3 is a side elevational cross-sectional view of the holder taken through the line 3-3 of FIG. 2 showing the retention features in the first position;

FIG. 4 is a side elevational cross-sectional view corresponding to FIG. 3 showing a specimen cup of a first dimension received within the supporting ring and retained therein by the retention features shifted or deformed or deflected to a second position;

FIG. 5 is a second side elevational cross-sectional view corresponding to FIG. 3 showing a specimen cup of a second dimension received within the supporting ring with the retention features in a third position;

FIG. 6 is a top plan view of a form of a holder including a frangible breakline for removing at least a part of the holder such as a handle portion;

FIG. 7 is a top plan view of a form of a holder similar to that of FIG. 6;

FIG. 8 is a side elevational view of a third form of a holder having a frangible breakline for removing at least a part of the holder;

FIG. 9 is a top plan view of a holder having arms defining an open boundary for receiving and supporting a cup therein, the arms in a general “V” shape and having inwardly extending portions for retaining the cup therein;

FIG. 10 is a top plan view of a holder similar to that of FIG. 9 wherein the arms are in a general arcuate or horseshoe shape;

FIG. 11 is a side elevational view of a holder having arms defining an open boundary for receiving and supporting a cup therein, the arms having upwardly extending portions for retaining the cup therein;

FIG. 12 is a top plan view of the holder of FIG. 11 showing the upwardly extending portions in contact with the cup therein;

FIG. 13 is an exploded side elevational view of a cup and holder system, the holder including a connector portion for connecting with a top edge and portion of the cup;

FIG. 14 is a fragmentary cross-sectional view of the system of FIG. 13 showing the connector portion joined with the top portion of the cup;

FIG. 15 is an exploded view of a cup and holder system, the cup and holder having cooperating structure for releasably coupling together in a generally horizontal manner;

FIG. 16 is an exploded view of a cup and holder system similar to that of FIG. 15, the cooperating structure instead releasably coupling in a generally vertical manner;

FIG. 17 is a side elevational view in cross-section showing a threaded connection between a holder ring and a cup, absorbent material being disposed proximate the juncture therebetween;

FIG. 18 is a side elevational view of a holder and cup system with a funnel;

FIG. 19 is a side elevational view of a holder including a funnel-shaped support ring for supporting a cup thereon;

FIG. 20 is a top plan view of a first form of a device for assisting in specimen collection wherein the device is supported by another fixture such as a toilet by terminal portions of the device;

FIG. 21 is a fragmentary view of a second form of terminal portions of the device of FIG. 20; and

FIG. 22 is a side elevational view of a holder including a handle and a ring showing a cross-sectional shape of the handle.

### DETAILED DESCRIPTION

Referring initially to FIGS. 1-5, a holder 10 for supporting a specimen cup 12 is shown having retention features in the form of flexible fingers 14 for permitting cups 12 of various sizes to be received and retained within a ring 16 of the holder 10. In this manner, a variety of sized cups 12 can be used with the holder 10, and the ring 16 need not have an opening or break therein in order to accommodate the different sized cups 12. Additionally, any cup 12 received within the ring 16 is pressed against around its periphery to retain and generally immobilize the cup 12 therewithin.

In a preferred form, the holder 10 is a unitary device formed integrally of polymeric material such as low or high-density polyethylene (LDPE or HDPE) or polyphenylene (PPE). In this manner, the holder 10 is simple and inexpensive to produce.

As can be seen, the holder 10 includes a grip portion 20, the ring 16 distally positioned from the grip 20, and an elongated handle 22 extending between and integrally formed with the grip 20 and ring 16. The thicknesses or dimensions of the grip 20 and ring 16 and handle 22 provide for rigidity in supporting and manipulating the ring 16 when the cup 12 is located therein and, in particular, when the cup 12 has been filled with a urine specimen.

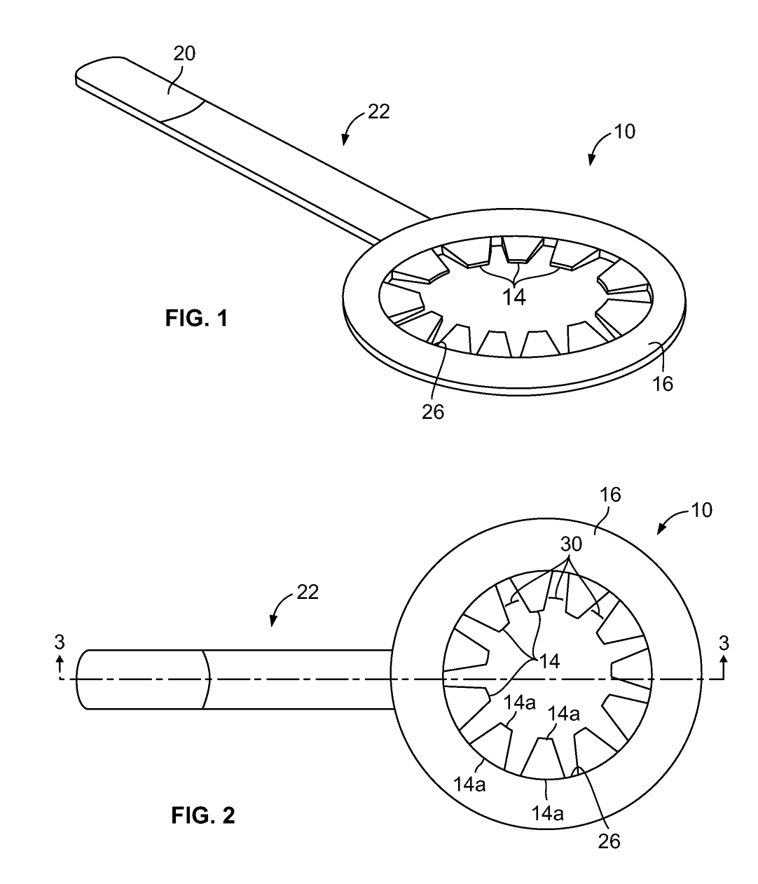

The fingers 14 have an initial or as-formed position shown in FIGS. 1-3 as generally extending radially inward from an inner periphery 26 of the ring 16. As best seen in FIG. 2, the fingers 14 are shaped somewhat as a frustum of a circular wedge or pie shape such that the fingers 14 have a base portion 14a secured (and preferably formed integral) with the ring inner periphery 26 such that the base portion 14a is arced to follow the shape of the inner periphery 26. The finger 14 extends from the base portion 14a to a tip 14b that, when viewed from the top as in FIG. 2, has an arcuate shape. The shape of the tips 14b in some forms may be such that, together, the tips 14b correspond to a circle co-centric with the circle defined by the ring 16 itself. This allows the tips 14b to generally correspond in shape to a cup 12 received therein, the cup 12 being concentrically received within the ring 16.

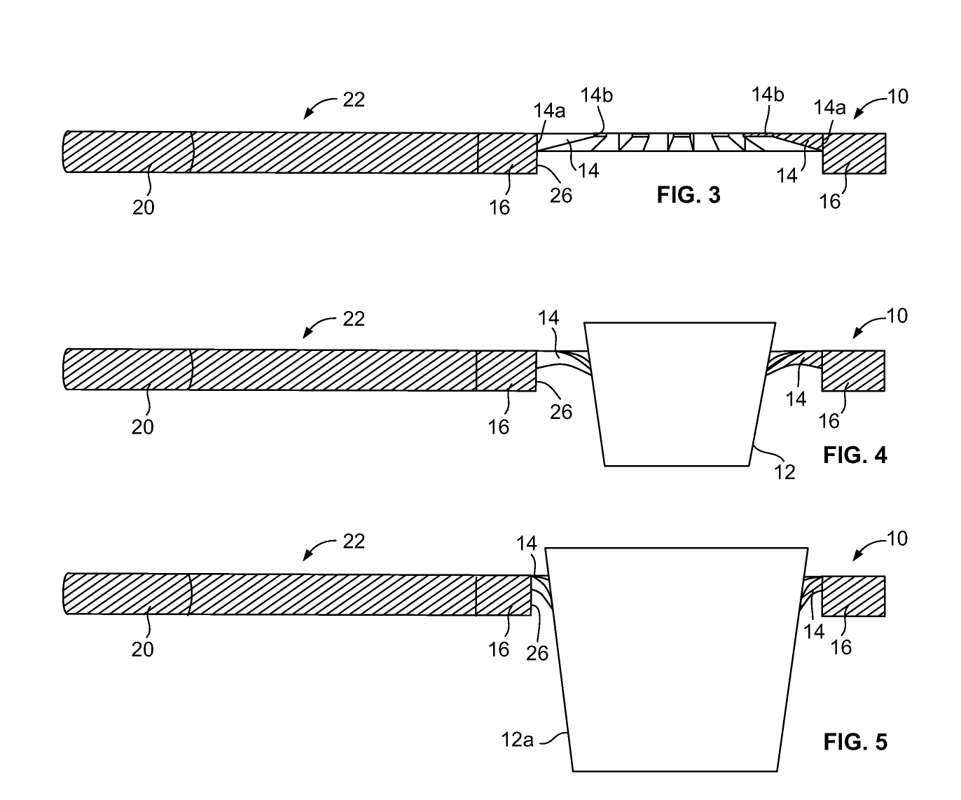

Best viewed in FIG. 3, the base portion 14a is thicker, in a generally vertical direction, than the tip portion 14b, though the finger 14 may be of generally uniform thickness. That is, the fingers 14 taper down from the base portion 14a to the tip portion 14b.

Comparing FIG. 3 with FIG. 4, the fingers 14 are pliable and, preferably, resiliently pliable so that the fingers 14 provide a retention force against the sides of the cup 12 received within the ring 16 and fingers 14. Specifically, advancement of the cup 12 into the ring 16 brings the cup 12 into contact with the tips 14b of the fingers 14. Continued advancement of the cup 12 deflects the fingers 14 downward to deform the fingers 14 from the initial position (i.e, that of FIG. 3) to a deflected position (that of FIG. 4) so that the fingers 14 resiliently press against the cup 12. As noted above, the fingers 14 are illustrated as tapering from the base portion 14a to the tip portion 14b, which allows a smaller cup 12 to receive a minimal amount of retention force from the fingers 14. In a form (not shown) wherein the fingers 14 are generally of uniform vertical thickness from the base portion 14a to the tip portion 14b, the connection between the finger 14 and the ring 16 could give rise to a stress concentrator: given the desire for inexpensive manufacturing and for disposability of the device, it is desired to permit relatively loose manufacturing tolerances and/or material creep, and the form (shown) having the tapered fingers 14 is believed to allow cheaper molds and cheaper manufacturing, while also providing less likelihood of the fingers 14 being broken from the ring 16.

Comparing FIGS. 4 and 5, it can be seen that the ring 16 and fingers 14 accommodate different sized cups 12. That is, the fingers 14 in FIG. 4 are displaced from the initial position of FIG. 3 to accommodate the cup 12. In FIG. 5, a larger cup 12a is received in the ring 16, which results in greater deflection of the fingers 14 from the initial position to a third deflected or deformed position.

It should be noted that the holder 10 is preferably formed of a homogenous material in a one-step molding process such as injection molding, though such need not be so. For instance, transfer molding may be used, but such is believed to be a more expensive operation. Additionally, the fingers 14 may be formed of a second material, such as a rubber or elastomeric material, joined with a first and more rigid material used for the ring. It should be noted further that the fingers 14 may extend from a finger ring (not shown) formed of the second material, and the finger ring is then joined (by molding or bonding or mechanically connecting, etc.) with the ring 16. The fingers 14 may alternatively simply be a continuous elastomeric ring (not shown) extending inward from the ring periphery 26 that expands and deflects downwardly when a cup 12 is received therein, pressing sleeve-like against the cup 12. However, while each of such forms is within the scope of the invention, each is also believed to be more expensive to manufacture.

It should also be noted that the fingers 14 accommodate some spillage. That is, as discussed above, it is difficult if not impossible to avoid any urine coming into contact with the outside of the cup 12. With the fingers 14, gaps 30 are defined between the fingers 14 which allows urine that may otherwise collect on the side of the cup 12 or at the juncture between the holder 10 and the cup 12 to simply pass. The urine is free to pass through the gaps 30 and drip off, such as into a toilet.

Accordingly, the holder 10 provides a number of benefits over the prior art. The holder 10 accommodates a variety of sized cups 12 while providing rigidity throughout the holder 10 and, in particular, the ring 16 thereof. The fingers 14 insure retention of a cup 12 with the ring 16, and the gaps 30 therebetween permit urine to pass through and drip off. Additionally, the holder 10 is designed to receive a cup 12, and to be separated from the cup 12 when the cup 12 is filled and sent to a laboratory for analysis of the urine sample.

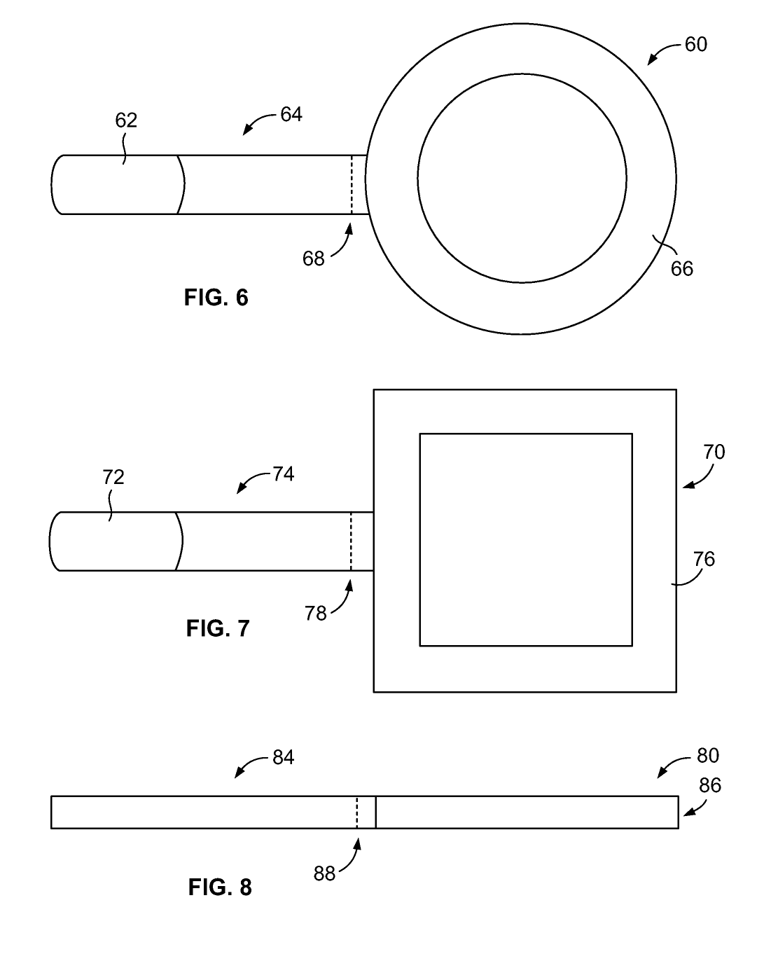

Turning now to FIGS. 6-8, other forms of specimen collection devices are illustrated. In FIG. 6, a holder 60 is shown having a grip 62, a handle 64, and a support in the form of a ring 66. A cup (not shown) may be received within the ring 66 (the ring 66 either including the retention features (i.e., fingers 14) described above or being as shown without such features) or integral therewith. The holder 60 includes a frangible breakline 68 so that the handle 64 or a majority thereof may be easily disconnected from the ring 66 when desired. FIG. 7 shows a holder 70 similar to the holder 60 of FIG. 6, though the holder 70 is shaped for a cup 12 having a non-circular shape, such as quadrilateral or square or frusto-pyramidal, as mere examples. Again, the holder 70 of FIG. 7 includes a frangible breakline 78 to remove the handle 74 from a support 76.

Viewing FIG. 8, a holder 80 is also shown having a frangible breakline 88. For the forms of FIGS. 6 and 7, the handles 64, 74 are removed by breaking them in a generally vertically direction. The holder 80 has its breakline 88 oriented so that the handle 84 is broken via lateral or horizontal motion relative to the ring 86. This allows the holder 80 to accommodate a greater weight of urine specimen in a cup 12 supported by the ring 86 while insuring that such weight does not inadvertently fracture or separate the breakline 88.

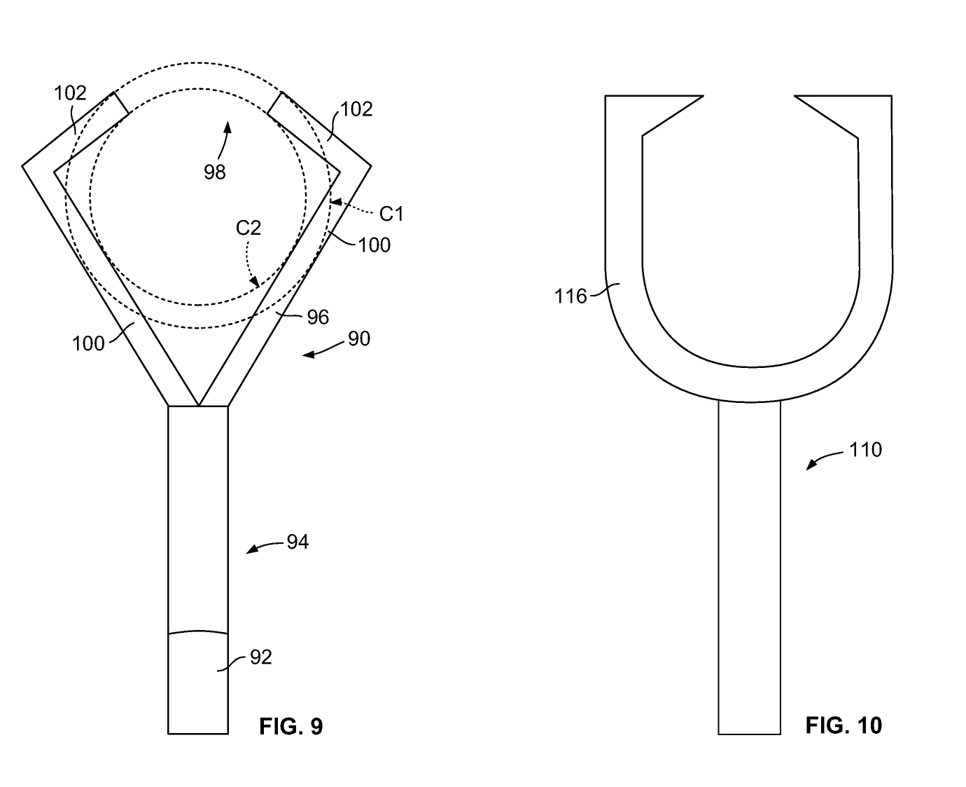

FIGS. 9-10 illustrate further forms of devices for assisting in specimen collection. FIG. 9 illustrates a holder 90 having a grip 92, an elongated handle 94, and a support 96 for receiving a cup 12 therein. However, unlike the previous forms, the support 96 does not define a closed boundary, instead having a large opening 98. The support 96 as shown includes first and second arms 100 extending from the handle 94 in a plane and defining a somewhat “V” shape. The arms 100 have terminal hands 102 thereon extending in at least a skew direction from the direction of the arms 100 so that a cup 12 may be received within and supported by the arms 100 and hands 102. The opening 98 is generally positioned opposite the handle 94 so that the ability of the support 96 to bear the weight of a filled specimen cup is dependent on the strength of the material(s) of the holder 90. The large opening 98 is generally at least a quarter of the periphery of a cup 12, though such will vary depending on the size of the cup 12, or a quarter of the boundary defined by the support 96. By providing the large opening 98, and by orienting the opening 98 opposite the handle 94 and grip 92, a user is able to determine without visual confirmation that the cup 12 is entering a urine stream at the opening 98 so that the holder 90 itself generally does not get wet. While it is not a specific intention to avoid getting the holder 90 wet with urine, as the holder 90 is intended to be disposable, it is the intention that any urine that comes into contact with the exterior of the cup 12 is to be permitted to drip off. With the large opening 98, the holder 90 itself does not resist or impede the ability of the urine to drip off therefrom. It should be noted that FIG. 9 shows general positions for a representative cup 12 in phantom lines, the circumference of a top edge of the cup being labeled C1 and a cup circumference that is generally co-planar with the holder 90 when the cup 12 is supported thereby being labeled C2. FIG. 10 shows a similar form of a holder 110 to that of FIG. 9, though having a support 116 that is generally horseshoe-shaped. It should be noted that a variety of shapes may be used within the scope of the invention to provide a large opening of predetermined orientation to allow the user to direct the holder and a cup supported thereon into a urine stream such that the large opening is easily aligned with the urine stream when entering the same.

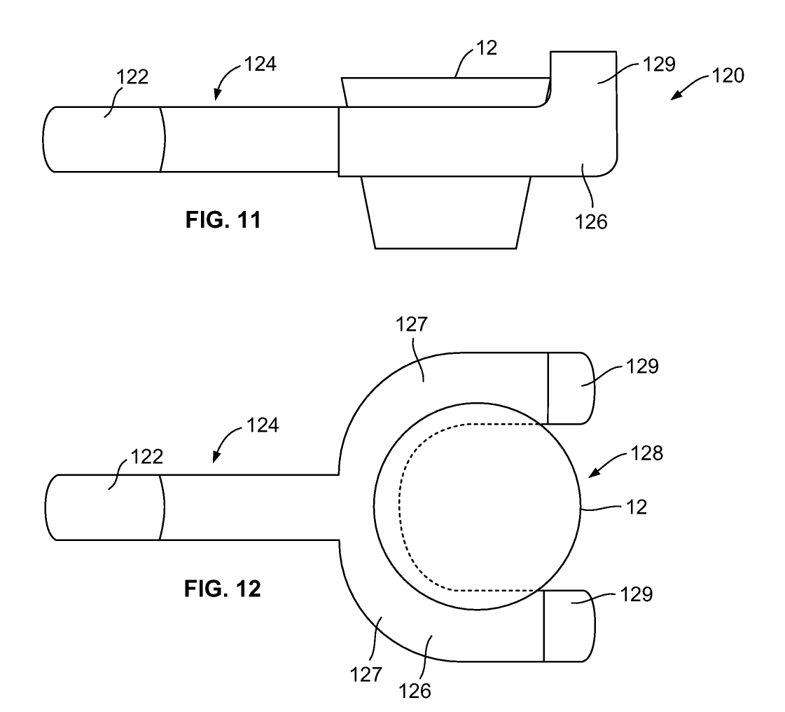

FIGS. 11 and 12 show devices similar to that of FIGS. 9 and 10. FIG. 11 illustrates a holder 120 having a grip 122, a handle 124, and a support 126 having a large opening 128, the opening 128 being opposite the handle 124 in the manner described for the holders 90 and 110. The support 126 includes arms 127 like the arms 100; however, the arms 127 include generally vertical hands 129 instead of the inwardly angled hands 102 of the holder 90, e.g. As can be seen, a cup 12 is supported at its sides within the arms 129 and is restricted from passing through the large opening 128 by the upstanding hands 129.

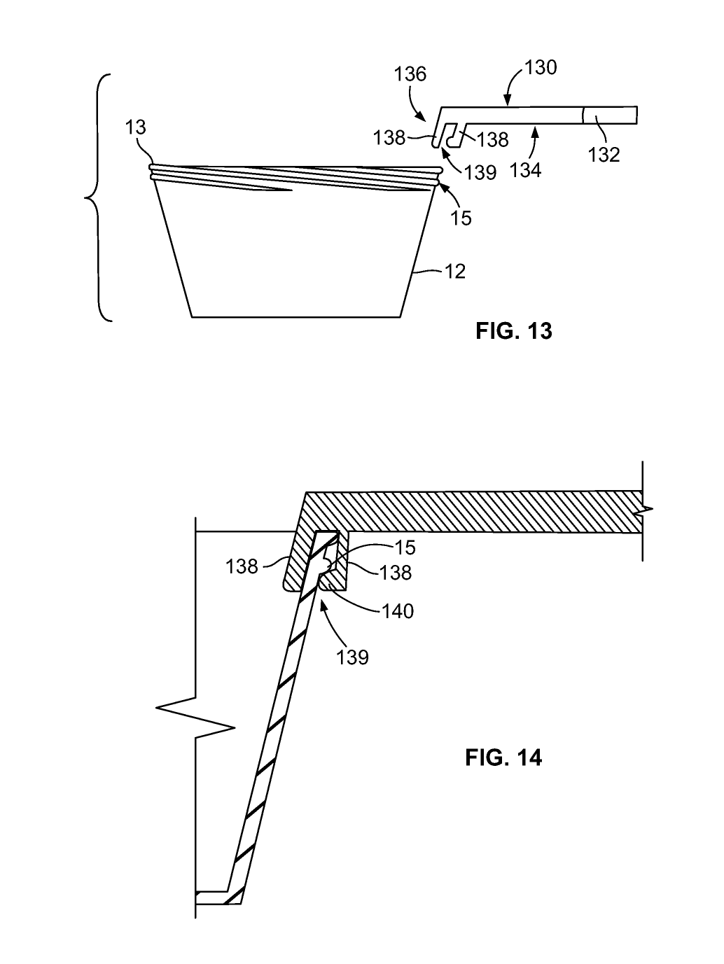

FIGS. 13 and 14 illustrate a holder 130 usable generally with any cup 12. The holder 130 includes a grip 132 and an elongated handle 134 extending therefrom to a connector 136. In general, specimen cups 12 having an upper edge 13 and may or may not have structure for receiving and securing a lid with the cup 12; in the event such structure is provided, it is generally either a lip for a snap-fit lid or threading for a threaded lid. The connector 136 of the present holder 130 may be in a variety of forms for joining with an upper portion of the cup 12 at the upper edge 13.

For instance, as illustrated in the detail of FIG. 14, the connector 136 includes a pair of connector walls 138 defining a channel 139 into which the cup 12 is advanced. The connector 136 is resiliently pressed onto the cup 12 (and the channel 139 is sized) so that, minimally, a friction fit is achieved.

Preferably, at least one of the walls 138 includes a gripping structure such as a rib 140 for providing a localized pressure area or region. In the event the cup 12 includes the described structure for receiving a lid, such as the cup rib or thread 15 illustrated in FIGS. 13 and 14, the rib 140 engages with such structure to retain the cup 12 and holder 130 together with an additional interference fit. In this manner, the holder 130 may be used with virtually any cup 12, and may easily be removed when the specimen cup 12 has been filled and is ready to be closed and sent to the laboratory for analysis.

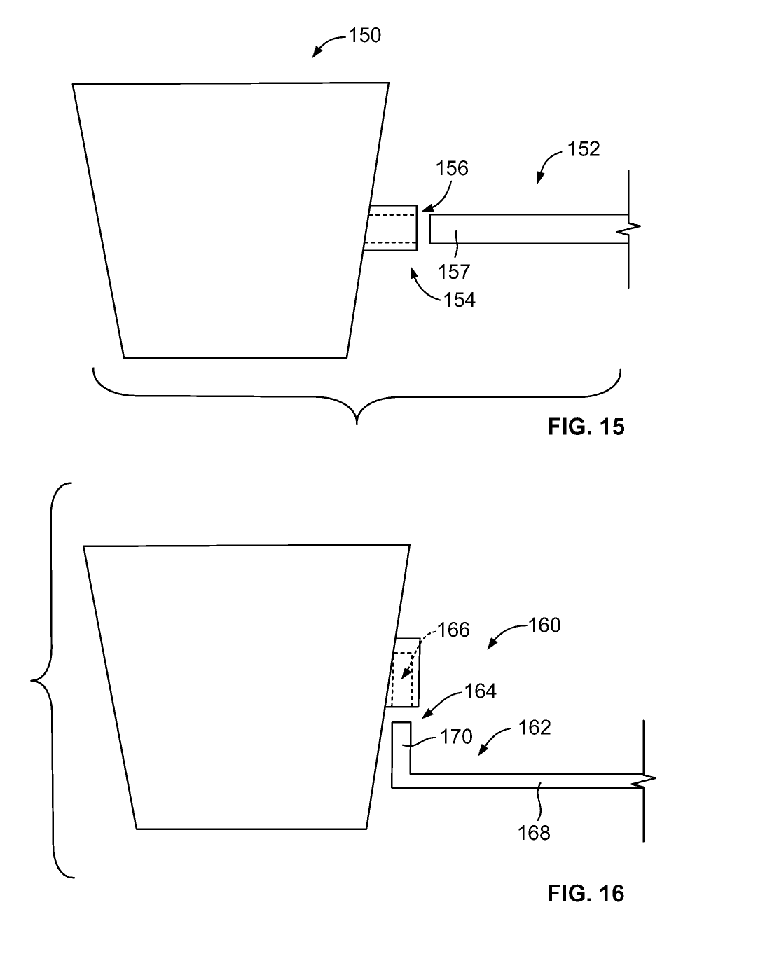

Turning now to FIGS. 15 and 16, cup and holder systems are shown. FIG. 15 shows a cup 150 and shows a holder 152 generally in the form of a rod, the cup 150 and holder 152 having cooperating structure 154 for removably connecting the holder 152 with the cup 150. Specifically, the structure 154 includes a post and socket joint, the cup 150 including a socket 156 while the holder 152 includes a post 157; obviously, such connection could easily be reversed.

Similar to that shown in FIG. 15, a cup 160 and holder 162 are illustrated in FIG. 16 having cooperating structure 164. However, in this form, the cup 160 has a vertically aligned (and preferably non-circular) socket 166, and the holder 162 includes an elongated handle 168 with an upstanding pin portion 170 that is received in (and preferably correspondingly shaped to) the socket 166. As such, the holder 162 can be released from the cup 160 simply by setting down the cup 160, such as on a specimen tray (not shown).

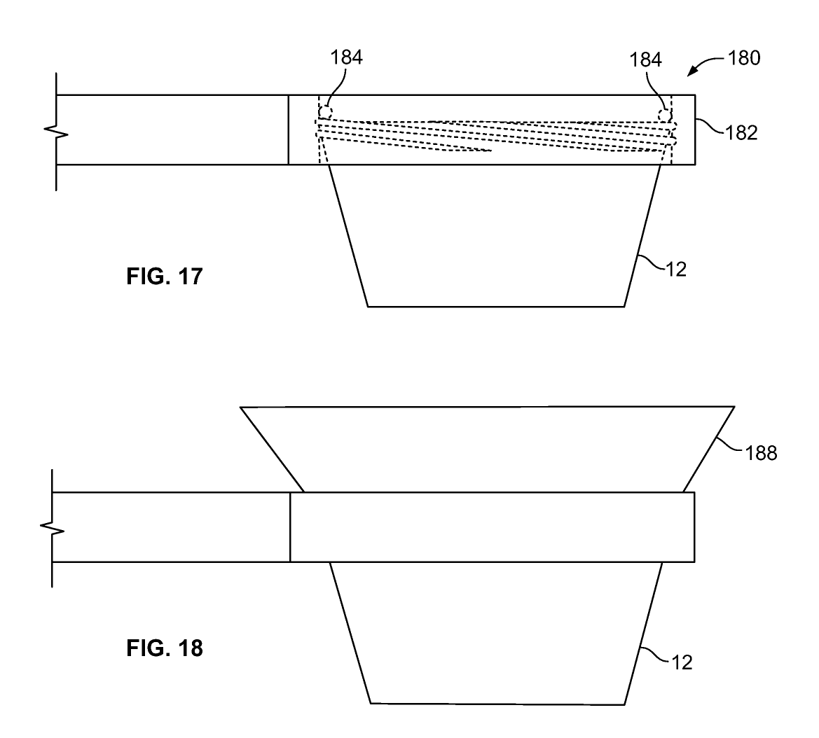

FIG. 17 illustrates a holder 180 for use with a threaded cup 12. The holder 180 includes a ring 182 having internal threading which mates with the threading on the cup 12. In this manner, a user is assured that the cup 12 will not become dislodged from the holder 180 during use. In a preferred form, absorbent material 184 is disposed at or around the junction between the holder 180 and the cup 12 to absorb and collect urine that is not received within the cup 12 but is received within the ring 182. In this manner, removal of the cup 12 from the ring 182 by unthreading the cup 12 does not result in spillage between the cup 12 and ring 182 from flowing down the cup 12 and onto a person's hands.

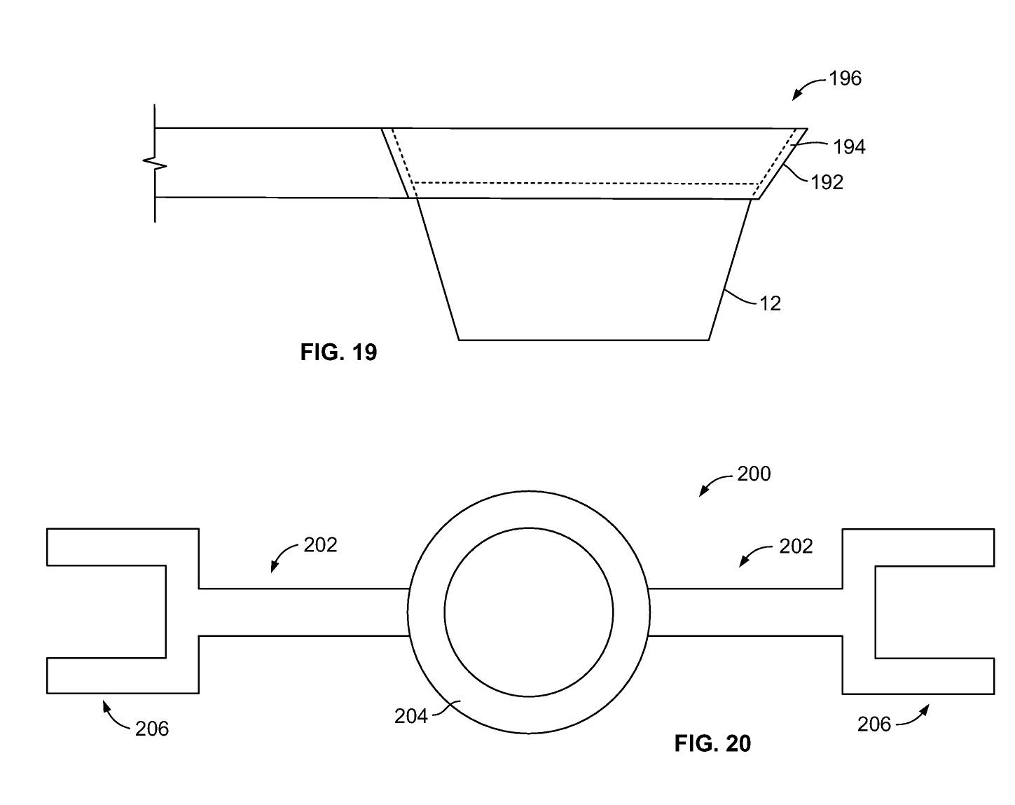

FIGS. 18 and 19 illustrate the use of funneling with forms of the devices described herein. In FIG. 18, one may simply provide a funnel 188 over the top of any cup and/or holder as described herein. In FIG. 19, a holder 190 is illustrated having a ring 192 that itself is a funnel having tapered sides 194. The holder 190 may be provided with fingers 14 and gaps 30 within the ring 192, as described above, so that urine does not collect at the interface between the holder 190 and a cup 12.

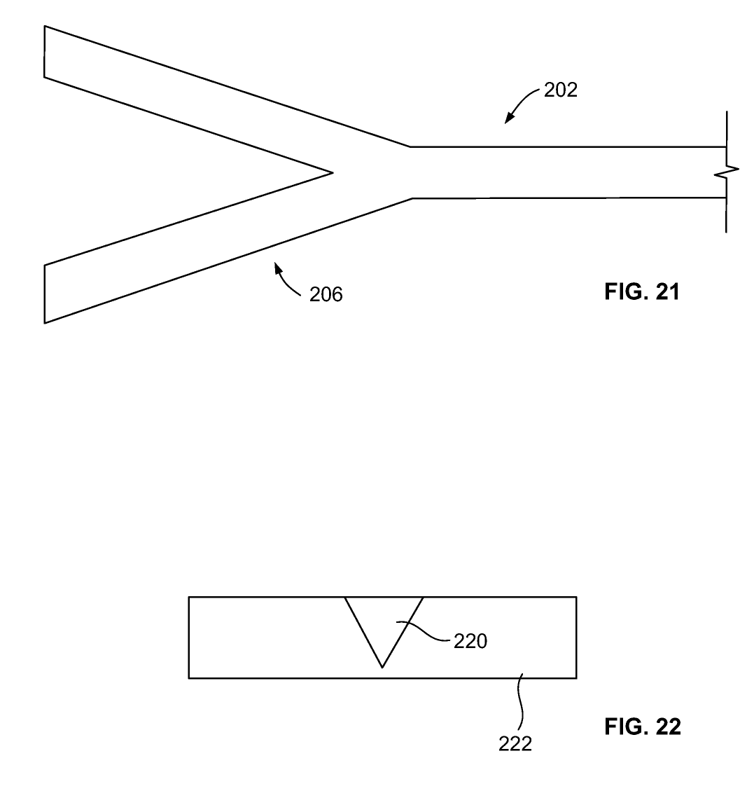

Turning now to FIGS. 20 and 21, additional devices for assisting in urine specimen collection are illustrated and described herein. While each of the above forms described herein generally contemplate a user and/or medical personnel manually holding and utilizing the devices, FIG. 20 illustrates a device that supported by the toilet itself. That is, FIG. 20 shows a system 200 including first and second arms 202 that extend across a toilet opening (not shown) such as by resting on a toilet seat or rim (not shown). In some forms, the arms 202 may be adjustable in radial orientation or in length for appropriate use with different sized or configured toilets. The arms 202 are joined by a support 204 therebetween, the support 204 either including or permitting receipt of a cup 12. In the form of FIG. 20, the arms 202 include terminal portions 206 that provide balance to the system 200, the terminal portions 206 being somewhat fork-like. However, the terminal portions 206 may be in a variety of shapes to generally provide more than a point contact with, e.g., the toilet, such as the “V” shaped terminal portions of FIG. 21.

It should be noted that, unless otherwise stated, for each embodiment described herein, the devices are generally considered to be somewhat planar, though the handles and the like may be angled from a plane of the support for holding the cup. It should also be noted that a connection between a handle and a ring, or elsewhere as desired or beneficial, is preferably designed with performance characteristics in mind. For instance, as can be seen in FIG. 22, a handle 220 has a cross-sectional shape/size/structure and joint with the ring 222 designed to allow the handle 220 to bear the weight of a filled specimen cup 12, without the ring 222 bending or breaking from the handle 220. Preferably, handle 220 is non-circular so that it provides a tactile feel or recognition to the user of the horizontal position of the cup 12. In other words, during use, a user that cannot see the cup 12 itself can recognize that an opening of the cup 12 is being maintained at a generally level plane so that urine directed therein is collected.

It should also be noted that, unless described as such, the relative scales of the portions of the devices as illustrated are not intended to be limiting, instead intending to be representational.

While the invention has been described with respect to specific examples including presently preferred modes of carrying out the invention, those skilled in the art will appreciate that there are numerous variations and permutations of the above described systems and techniques that fall within the spirit and scope of the invention as set forth in the appended claims.

What is claimed is:

1. A device for utilizing a specimen collection cup, the device comprising:
a handle portion;
a support portion connected with the handle portion and defining a closed boundary; and
retention features;
wherein the support portion is configured to receive cups of varying sizes within the closed boundary, and wherein the retention features provide a resilient retention force to the cup received therein.

2. The device of claim 1 wherein the support portion defines a circular closed boundary and is generally ring-shaped.

3. The device of claim 1 wherein the retention features include a resiliently deformable portion extending inwardly into the closed boundary.

4. The device of claim 3 wherein the resiliently deformable portion includes a plurality of fingers.

5. The device of claim 4 wherein the fingers are formed integral with the support portion.

6. The device of claim 4 wherein the fingers taper from a base portion at the support portion to a tip portion to provide variable retention force to the varying sized cups.

7. The device of claim 4 wherein the support portion is in the form of a ring, the fingers have an arcuate base connected with the support portion, and the fingers have an arcuate tip portion for contacting and providing retention force against a cup received within the support portion.

8. The device of claim 1 wherein the device is formed integral.

9. A holder for supporting and manipulating a specimen cup for collecting urine, the holder comprising:
a support portion for supporting a specimen cup;
an elongated handle extending from the support portion; and
a frangible breakline for removing at least a portion of the handle from the support portion.

10. The holder of claim 9 wherein the breakline is frangible by applying a lateral force.

11. The holder of claim 9 wherein the breakline is frangible by applying a vertical force.

12. The holder of claim 9 wherein the holder further includes a specimen cup.

13. The holder of claim 12 wherein the specimen cup is integral with the support portion.

14. A holder for supporting and manipulating a specimen cup for collecting urine, the holder comprising:
an elongated handle for directing the holder; and
a rigid support portion connected with the handle, wherein the support portion includes first and second arms defining an open boundary including an opening, the open boundary for receiving cups therein, and the arms including terminal portions for retaining the cups therein.

15. The holder of claim 14 wherein the opening is opposite the handle.

16. The holder of claim 15 wherein the opening is at least a quarter of the boundary.

17. The holder of claim 14 wherein the arms are generally planar and the terminal portions thereof are angled to prevent cups from passing through the opening.

18. The holder of claim 14 wherein the terminal portions extend upwardly from a general plane of the arms, the terminal portions preventing cups from passing through the opening.

19. A holder for supporting and manipulating a specimen cup for collecting urine, the holder comprising:
an elongated handle for directing the holder; and
a connector formed on a distal end of the holder, the connector releasably securable with a portion of a specimen cup.

20. The holder of claim 19 wherein the connector includes a pair of walls defining a channel, and a top portion of the cup is receivable between the walls.

21. The holder of claim 20 wherein the walls exert a friction force against a cup received therebetween, the friction force retaining the cup with the holder.

22. The holder of claim 21 wherein the walls include securing structure for retaining the connector with the cup.

23. The holder of claim 22 wherein the securing structure is at least a riblike structure for pressing into the cup.

24. The holder of claim 22 wherein the securing structure is at least a riblike structure for providing an interference fit with a portion of the cup.

25. The holder of claim 19 wherein the connector and cup having corresponding mating structure forming a post-and-socket joint.

26. The holder of claim 25 wherein the joint includes a horizontal socket and a post received horizontally therein.

27. The holder of claim 25 wherein the joint includes a vertical socket and a post received vertically therein.

28. A holder for supporting and manipulating a specimen cup for collecting urine, the holder comprising:
an elongated handle for directing the holder;
a support portion for receiving a specimen cup; and
a funnel positionable over and into a specimen cup received by the support portion for directing urine into the cup.

29. A holder for supporting and manipulating a specimen cup for collecting urine, the holder comprising:
an elongated handle for directing the holder;
a support portion for receiving a specimen cup; and
a threaded engagement between the support portion and the specimen cup.

30. The holder of claim 29 further including an absorbent material disposed proximate the threaded engagement.

31. A holder for supporting and manipulating a specimen cup for collecting urine, the holder comprising:
an elongated handle for directing the holder;
a support portion for receiving a specimen cup,
wherein the support portion includes a generally funnel-shape for directing urine into a cup supported thereby.

32. The holder of claim 31 wherein the support portion includes a plurality of inwardly extending retention fingers for supporting a cup therein, the fingers separated by gaps permitting urine to pass therethrough.

33. A holder for supporting and manipulating a specimen cup for collecting urine, the holder comprising:
a support portion for supporting a specimen cup; and
first and second arms extending from the support portion and supportable by a toilet.

34. The holder of claim 33 wherein the first and second arms include widened end portions for resisting movement of the holder.

35. The holder of claim 34 wherein the widened end portions include fork-like portions.

36. The holder of claim 33 wherein the support portion includes the specimen cup.
